(19) United States
(12) Patent Application Publication
(10) Pub. No.: US 2013/0060155 A1
(43) Pub. Date: Mar. 7, 2013

(54) EAR-CARRYING HEART BEAT DETECTION DEVICE

(71) Applicant: Yi-Lun Chen, New Taipei City (TW)

(72) Inventor: Yi-Lun Chen, New Taipei City (TW)

(73) Assignee: BION INC., NEW TAIPEI CITY (TW)

(21) Appl. No.: 13/592,676

(22) Filed: Aug. 23, 2012

(30) Foreign Application Priority Data
Sep. 6, 2011 (TW) 100216639

(51) Int. Cl. A61B 5/026

(52) U.S. Cl. 600/504

(57) ABSTRACT

The present invention relates to an ear-carrying heart beat detection device, which includes a device body, an attaching structure, an ear clip, an extension assembly, a control circuit and a wrist watch unit. The attaching structure is coupled to the device body. The ear clip includes a blood flowrate sensor to detect variation of blood flowrate through a user's earlobe. The extension assembly adjusts the relative distance of the ear clip with respect to the device body. The control circuit is arranged inside the device body and the control circuit transmits a heart beat signal to a wrist watch unit in a wireless manner to allow the user to inspect the condition of heart beat of his or her body and to adjust the time period and style of exercise according to the heart beat information.

### BACKGROUND OF THE INVENTION

1. Field of the Invention

The present invention relates to an ear-carrying heart beat detection device, and in particular to a device comprising an ear clip that comprises a coupling member to couple to a bar and the bar is rotatably mounted to a device body to enable adjustment of a relative angle between the bar and the device body so as to provide various angles suiting various applications.

2. The Related Arts

The progress of medical technology and equipment makes people taking heath care more seriously and the life of human being is universally extended. This significantly increases the population of aged people. However, the trained medical personnel is not far behind the rapid increase of the aged people, so that technology is the only feasible solution that allows the health conditions of the aged people to be generally monitored with various monitoring devices and assisting devices in order to eliminate unneeded waste and ineffective use of medical resources.

On the other hand, as mentioned above, people are paying more attention to health care. Consequently, various assisting devices are available in the market for monitoring body health condition. Among these devices, heart beat and pulse detection devices provide a function of detecting the conditions of heart beats and pulses and are widely used in the fields of medical equipment and exercise equipment.

A heart beat and pulse detection device can be used in combination with exercise equipment, whereby when a user is taking an exercise, the speed of heart beat and pulse can be detected by the heart beat and pulse detection device.

Various patents are available in the technical field of heart beat and pulse detection devices. Among these patents, a published patent, No. 20040665, discloses a structure of a transmitter of pulse watch. Such a structure transmits a heart beat signal to a pulse watch to display cardiopulmonary information for subsequent use by a user and comprises a device body in which a signal transmitter is mounted to transmit the heart beat signal, an insulation chest band, a coupling shaft arranged between the chest band and the device body to take a stretching force between the device body and the chest band, and a conductive chest band arranged on the insulation chest band to directly contact the chest of the user for detecting the heart beat signal and transmitting the signal to the signal transmitter.

Another known design of conventional heart beat and pulse detection devices sets a contact electrode on a top surface or a side surface of a watch-like device body, whereby a user may place both his or her hand in contact with the contact electrodes respectively to detect the conditions of heart beat and pulse.

Taiwan Utility Model No. 501747 discloses a wrist watch with heart beat/pulse detection, wherein a transducer lens is mounted on a body of the watch and a core received inside the water body is provided with a circuit control unit that is capable of detecting heart beat and pulse. The circuit control unit emits an infrared ray to detect variation of blood concentration of a human body in order to identify the number of beating of heart or pulse. An optic control circuit is also adopted to detect if the user has placed his or her fingers contacting the transducer lens in order to activate the counting function of the control circuit unit. As such, the number of beating of heart/pulse can be precisely detected and false operation caused by external noise can be eliminated. Besides the heart beat detection devices discussed above, a heart beat detection device is recently made available, which comprises an attaching structure for attaching to an ear of a user and an ear clip is provided for clamping the earlobe of the user. The ear clip comprises a blood flowrate sensor, whereby the condition of heart beat of the user can be identified through the detection of the variation of blood flowrate through the earlobe of the user by the blood flowrate sensor.

However, such a known ear-carrying heart beat detection device discussed above comprises an attaching structure that is fixed and does not suit every individual user. Further, due to being fixed, the relative position and angle between components thereof cannot be adjusted in a fine manner and this often cause discomfort when the device is used by the user, In addition, the conventional ear-carrying heart beat detection device will easily generate shaking signals when the user is taking an exercise. This affects measurement of heart beat information of the user and the heart beat information so detected can be incorrect, making is valueless for reference. Further, the conventional ear-carrying heart beat detection device simple functions of detecting and storing heart beat information and is not capable of transmitting the information to a wrist watch component. And, being incapable of fine adjustment of relative position and angle is an actually existing drawback and may make the user feel uncomfortable in use.

The present invention uses an extendable component to fix the ear clip so that the heart beat information of a user may not be affected by shaking signal generated by shaking of the ear clip when the user is taking an exercise, whereby correct heart beat information can be detected for reference by the user. Further, the present invention comprises a replaceable collar clip that can be used interchangeably,

Since the conventional ear-carrying heart beat detection device is in capable of transmitting data to a wrist watch component, the user cannot get the heart beat information immediately and is not allowed to adjust the style and period of time of the exercise he or she is taking according to the heart beat information so obtained.

### SUMMARY OF THE INVENTION

The present invention is made to address the issue that the conventional ear-carrying heart beat detection device, when worn on a user, is difficult to adjust to suit different users and is also hard for a user to do fine adjustment of the wearing location, so that the user may feel uncomfortable and improvement is desired. An object of the present invention is to provide an ear-carrying heart beat detection device that has a simple structure and is durable.

The present invention comprises an ear clip that comprises a coupling member to couple to a bar. The bar has a pivot joint that couples to a pivot pin formed on the device body to enable of adjustment of relative angle between the bar and the device body to provide the needs for different angles by different users and also for allowing a user to conduct a fine adjustment of position according to a specific situation, such as jogging, bicycle riding, or other exercises, which may otherwise generates a shaking signal that affects the detection of the heart beat information, making the detected heart beat information of the user incorrect and thus valueless for reference. With the bar holding the ear clip, the shaking signal caused by exercise taken by the user and correct and valuable heart beat information can be obtained for reference.

The ear-carrying heart beat detection device according to the present invention comprises a device body, an attaching structure, an ear clip, an extension assembly, a control circuit, and a wrist watch unit. The device body comprises a first coupling section and a first pivot joint section.

In the ear-carrying heart beat detection device, the attaching structure comprises an attaching section and a coupling component. The coupling component is coupled to the first coupling section of the device body to have the attaching structure coupled to the device body. The attaching section can be worn on a selected position by a user

The ear clip comprises a clamping section, a signal transmission wire, and a second coupling section. The clamping section comprises a blood flowrate sensor mounted thereto. The signal transmission wire is connected to the device body, whereby when the clamping section clamps on an earlobe of the user, the blood flow sensor detects the variation of blood flowrate through the earlobe of the user to thereby identify the condition of heart beat of the user that is transmit through the signal transmission wire to the device body.

The extension assembly comprises an extendable bar. The extendable bar comprises a second pivot joint section and the extendable bar is coupled to the second coupling section of the ear clip. Further, the extendable bar is coupled to the first pivot joint section of the device body with the second pivot joint section.

As such, when the attaching section is worn by a user on an ear with the clamping section of the ear clip clamping on a selected position of the earlobe, the user is allowed to rotate or move the ear clip with respect o the extendable bar or to rotate the extendable bar with respect to the first pivot joint section of the device body to achieve the purpose of fine adjustment of position or angle and provides the advantages of easy operation and making the user comfortable.

In the ear-carrying heart beat detection device described above, the first pivot joint section can be a pivot pin and correspondingly, the second pivot joint section of the extendable bar comprises a pivot hole rotatably jointing the pivot pin. The pivot hole can be formed in an end of the extendable bar. As such, the extendable bar, when subjected to an external force, may be rotated with respect to the device body so as to provide an advantage of easy operation for fine adjustment of the angle therebetween.

In the ear-carrying heart beat detection device so described, the extendable bar has an opposite end forming a stop. This arrangement prevents the extendable bar and the ear clip from separating from each other.

In the ear-carrying heart beat detection device described above, the second coupling section of the ear clip comprises a clamp. The clamp rotatably clamps on the extendable bar, whereby with an external properly applied, the clamp is movable or rotatable with respect to the extendable bar for fine adjustment of the relative angle and relative position between the two, thereby achieving an advantage of easy operation and comfortableness.

In the ear-carrying heart beat detection device described above, the first coupling section of the device body and the coupling component of the attaching structure are respectively provided with mateable positioning elements. The positioning elements are mateable toothed rings. As such, angular adjustment and positioning of the attaching structure can be carried out stably.

The control circuit is arranged inside the device body. The control circuit comprises a heart beat detection circuit and a wireless transmission circuit. The heart beat detection circuit is connected via the signal transmission wire to the blood flowrate sensor to receive a blood flowrate signal generated by the blood flowrate sensor and to generate a heart beat signal in response thereto.

The wireless transmission circuit is connected to the heart beat detection circuit to receive the heart beat signal and transmit the heart beat signal in a wireless form to a wireless receiver circuit of the wrist watch unit.

The wrist watch unit is worn on the wrist of the user. The wrist watch unit comprises a wireless receiver circuit that receives a heart beat signal transmitted from a wireless transmission circuit, a processing unit that is connected to the wireless receiver circuit, a display unit that is connected to the processing unit to display the heart beat information, and a memory unit that is connected to the processing unit to store the heart beat information.

It is appreciated from the above description that the present invention provides an ear-carrying heart beat detection device, which transmits detected heart beat information of a user to a wrist watch unit and is suitable to different users, various style of wearing, and also allows a user to effect fine adjustment of angle and position of use according to different situations of use, so that it is comfortable and easy to operate. The structure is simple and durable, has a low cost, and allows a user to inspect his or her own physiological data in addition to detecting the heart beat information of the user, so that the user may adjust the time period and style of exercise according to the understanding of the current exercise condition of the user himself or herself.

### BRIEF DESCRIPTION OF THE DRAWINGS

The present invention will be apparent to those skilled in the art by reading the following description of preferred embodiments of the present invention, with reference to the attached drawings, in which:

FIG. 1 is an exploded view of the present invention;

FIG. 2 is a perspective view showing the present invention with all components assembled;

FIG. 3 is a schematic view illustrating the operation of the present invention;

FIG. 4 is a schematic view illustrating the ear-carrying heart beat detection device according to the present invention put on an ear of the user;

FIG. 5 is a schematic view of a circuit according to the present invention;

FIG. 6 is a schematic view using an example of application of the present invention;

FIG. 7 is a schematic view showing a second embodiment according to the present invention;

FIG. 8 is a perspective view showing a third embodiment according to the present invention;

FIG. 9 is a schematic view illustrating n application of the third embodiment of the present invention;

FIG. 10 is a plot showing shaking signal generated by a conventional ear clip heart beat detection device when a user is taking an exercise; and

FIG. 11 is a plot showing the shaking signals occurring when a user is taking exercise are eliminated according to the present invention.

### DETAILED DESCRIPTION OF THE PREFERRED EMBODIMENTS

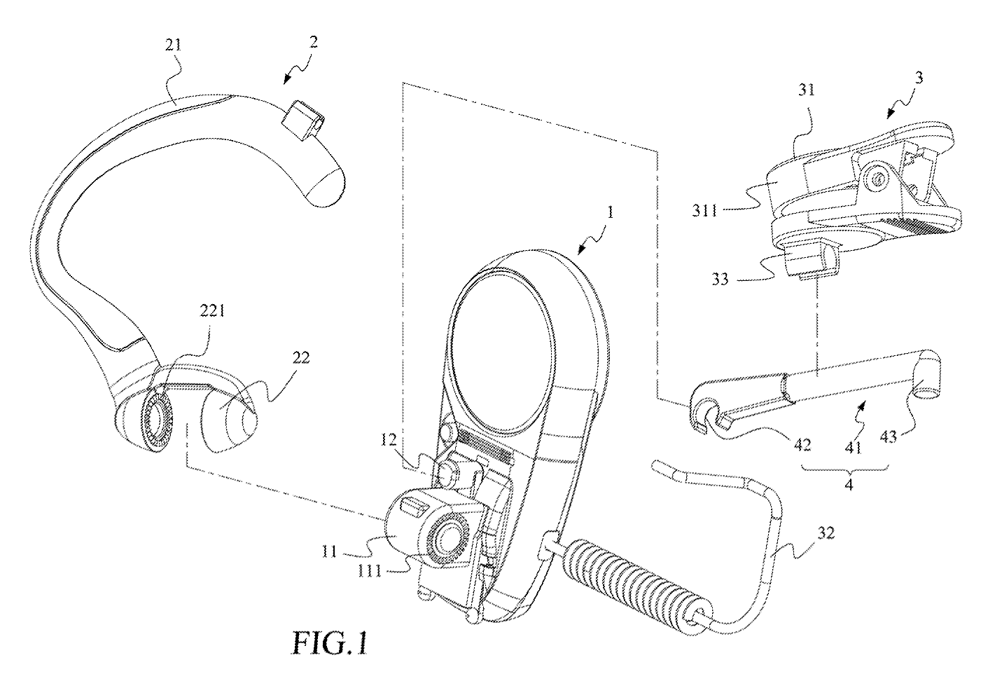

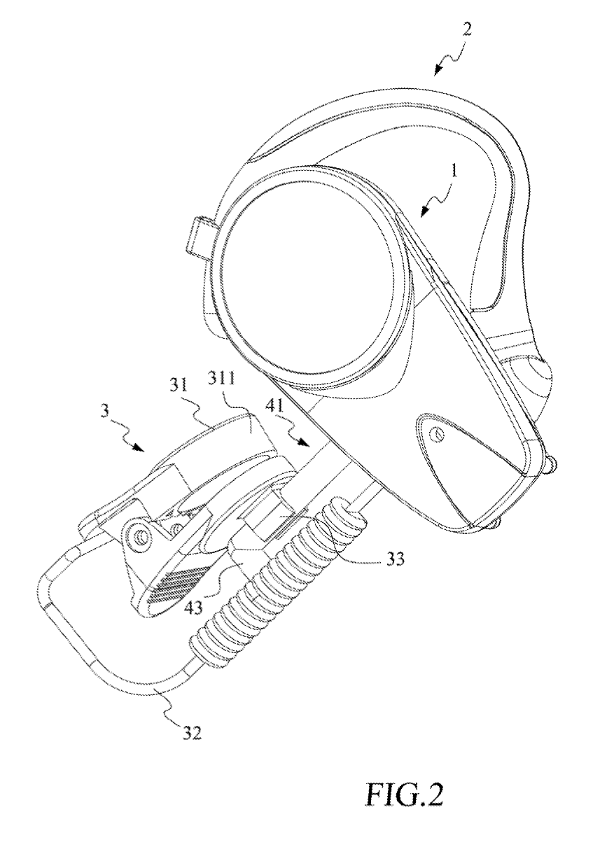

With reference to the drawings and in particular to FIGS. 1 and 2, FIG. 1 is an exploded view of the present invention and FIG. 2 is a perspective view showing the present invention with all components assembled. The present invention provides an ear-carrying heart beat detection device, which comprises a device body 1, an attaching structure 2, an ear clip 3, an extension assembly 4, a control circuit 6, and a wrist watch unit 7, of which the structures will be described in detail as follows:

The device body 1 comprises a first coupling section 11 and a first pivot joint section 12.

The attaching structure 2 comprises an attaching section 21 and a coupling component 22. The coupling component 22 functions to couple to the first coupling section 11 of the device body 1 so as to couple the attaching structure 2 to the device body 1.

The ear clip 3 comprises a damping section 31, a signal transmission wire 32, and a second coupling section 33. The clamping section 31 comprises a blood flowrate sensor 311 mounted thereto and connected with the signal transmission wire 32 to detect a blood flowrate signal from an earlobe of a user. The signal transmission wire 32 is connected to the device body 1.

The extension assembly 4 is connected between the ear dip 3 and the device body 1 whereby the relative distance of the ear clip 3 with respect to the device body 1 is adjustable with the extension assembly 4. The extension assembly comprises an extendable bar 41, The extendable bar 41 comprises a second pivot joint section 42 and the extendable bar 41 is coupled to the second coupling section 33 of the ear clip 3. Further, the extendable bar 41 is coupled to the first pivot joint section 12 of the device body 1 with the second pivot joint section 42.

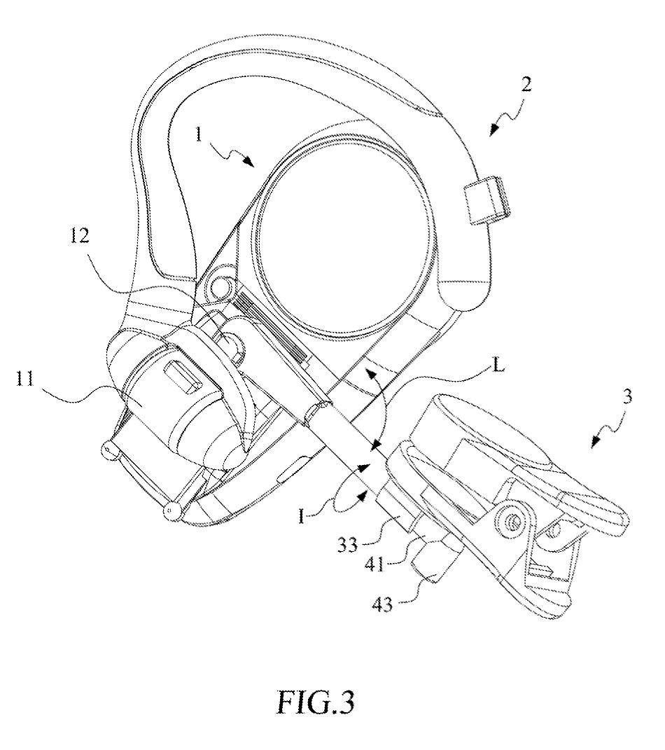

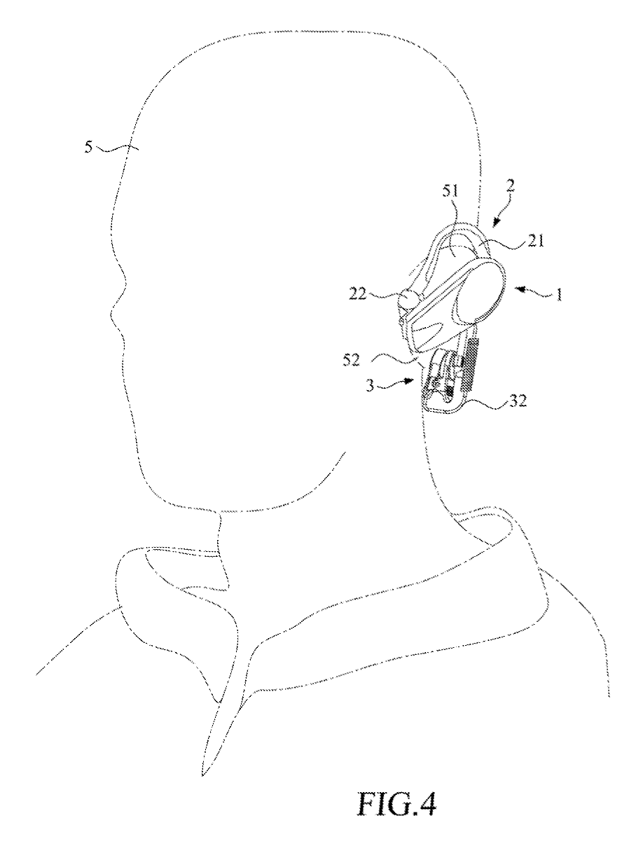

Referring to FIGS. 3 and 4. FIG. 3 is schematic view illustrating the operation of the present invention for adjusting angle according to the present invention and FIG. 4 is a schematic view illustrating the ear-carrying heart beat detection device according to the present invention put on an ear of the user to demonstrate the application of the present invention to the user.

The attaching section 21 is structured to allow the user 5 to wear at selected position on the ear 51. The ear clip 3 is secured in position by having the clamping section 31 clamping the earlobe 52 of the user 5, where the blood flow sensor 311 can detect variation of blood flowrate through the earlobe of the user thereby identifying the condition of heart beat of the user, which is then transmitted through the signal transmission wire 32 to the device body 1. Thus, when the attaching section 21 is put on the ear 51 by the user 5 with the clamping section 31 of the ear clip 3 clamping a selected position on the earlobe 52, the user is allowed to move or rotate the ear clip 3 with the extendable bar 41 in direction I or to rotate the extendable bar 41 with respect to the first pivot joint section 12 of the device body 1 in direction L in order to effect fine adjustment and to achieve the advantages of easy operation and making the user comfortable.

In the ear-carrying heart beat detection device described above, the first pivot joint section 12 can be a pivot pin and correspondingly, the second pivot joint section 42 of the extendable bar 41 comprises a pivot hole rotatably jointing the pivot pin (namely the first pivot joint section 12). The pivot hole (namely the second pivot joint section 42) can be formed in an end of the extendable bar 41. As such, the extendable bar 41, when subjected to an external force, may be rotated with respect to the device body 1 in the direction L so as to provide an advantage of easy operation for fine adjustment of the angle therebetween.

In the ear-carrying heart beat detection device so described, the extendable bar 41 has an opposite end forming a stop 43. This arrangement prevents the extendable bar 41 and the ear clip 3 from separating from each other.

In the ear-carrying heart beat detection device described above, the second coupling section 33 of the ear clip 3 comprises a clamp. The clamp (namely the second coupling section 33) rotatably clamps on the extendable bar 41, whereby with an external properly applied, the clamp is movable or rotatable with respect to the extendable bar 41 in the direction I for fine adjustment of the relative angle and relative position between the two, thereby achieving an advantage of easy operation and comfortableness.

In the ear-carrying heart beat detection device described above, the first coupling section 11 of the device body 1 and the coupling component 22 of the attaching structure 2 are respectively provided with mateable positioning elements 111 and 221. The positioning elements 111 and 221 are mateable toothed rings. As such, angular adjustment and positioning of the attaching structure can be carried out stably.

The above description given to the ear-carrying heart beat detection device according to the present invention shows the present invention is suitable for various individual users, various styles of wearing, and provide a user with the capability of fine adjustment of angle and position of use with easy operation and comfortableness. In addition, the structure is simple and durable, has a low cost, and allows a user to inspect his or her own physiological data in addition to detecting the heart beat information of the user, so that the user may adjust the time period and style of exercise according to the understanding of the current exercise condition of the user himself or herself.

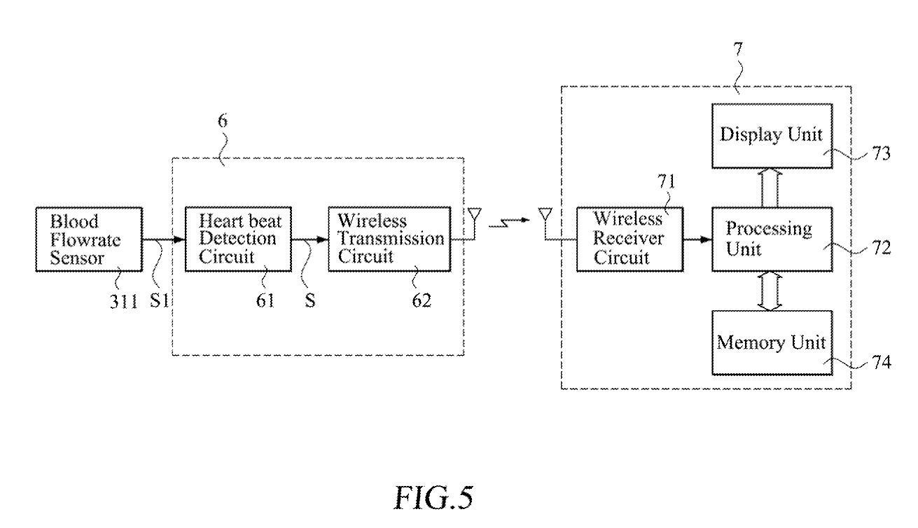

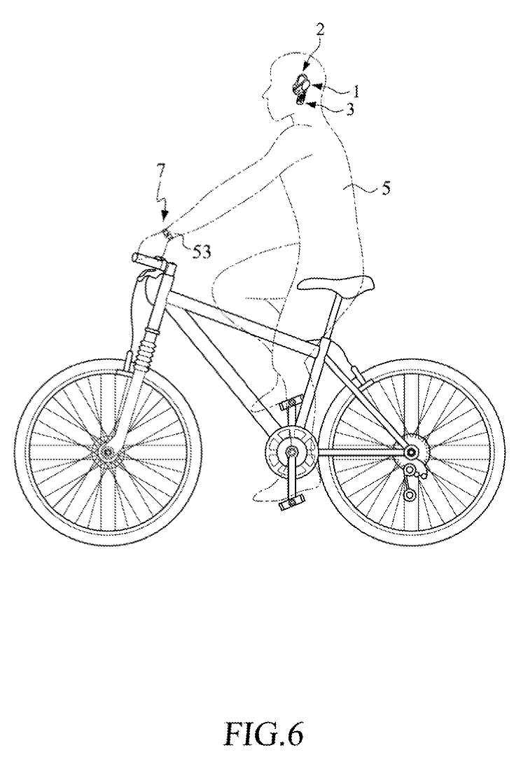

Referring to FIGS. 5 and 6, FIG. 5 is a schematic view of a circuit according to the present invention and FIG. 6 is a schematic view using an example of application of the present invention. The control circuit 6 is arranged inside the device body 1. The control circuit 6 comprises a heart beat detection circuit 61 and a wireless transmission circuit 62.

The heart beat detection circuit 61 is connected via the signal transmission wire 32 to the blood flowrate sensor 311 to receive a blood flowrate signal S1 generated by the blood flowrate sensor 311 and to generate a heart beat signal S in response thereto.

The wireless transmission circuit 62 is connected to the heart beat detection circuit 61 to receive the heart beat signal S and transmit the heart beat signal Sin a wireless form to a wireless receiver circuit 71 of the wrist watch unit 7.

The wrist watch unit 7 is worn on the wrist 53 of the user 5. The wrist watch unit comprises a wireless receiver circuit 71, a processing unit 72, a display unit 73, and a memory unit 74.

The wireless receiver circuit 71 receives the heart beat signal S transmitted from the wireless transmission circuit 62 and in turn conveys the heart beat signal S to the processing unit 72 for subjecting the heart beat signal S to signal processing.

The processing unit 72 is connected to the wireless receiver circuit 71 to receive the heart beat signal S generated by the heart beat detection circuit el which after being subjected to signal processing is fed to the display unit 73 to allow the user 5 to read the heart beat information and to store the heart beat information in the memory unit 74.

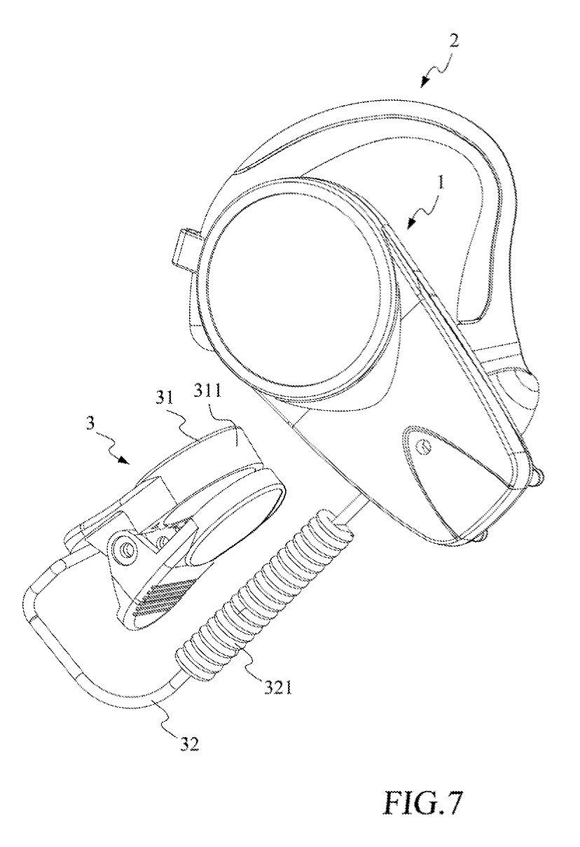

Referring to FIG. 7, the structure of a second embodiment according to the present invention is shown As shown in the drawing, the ear clip 3 comprises a clamping section 31, a signal transmission wire 32, and a second coupling section 33. The clamping section 31 comprises a blood flowrate sensor 311 mounted thereto and connected with the signal transmission wire 32 to detect the blood flowrate signal from the earlobe of the user. The signal transmission wire 32a comprises at least one extendable section 321, whereby the relative distance of the ear clip 3 with respect to the device body 1 can be adjusted with the extendable section 321.

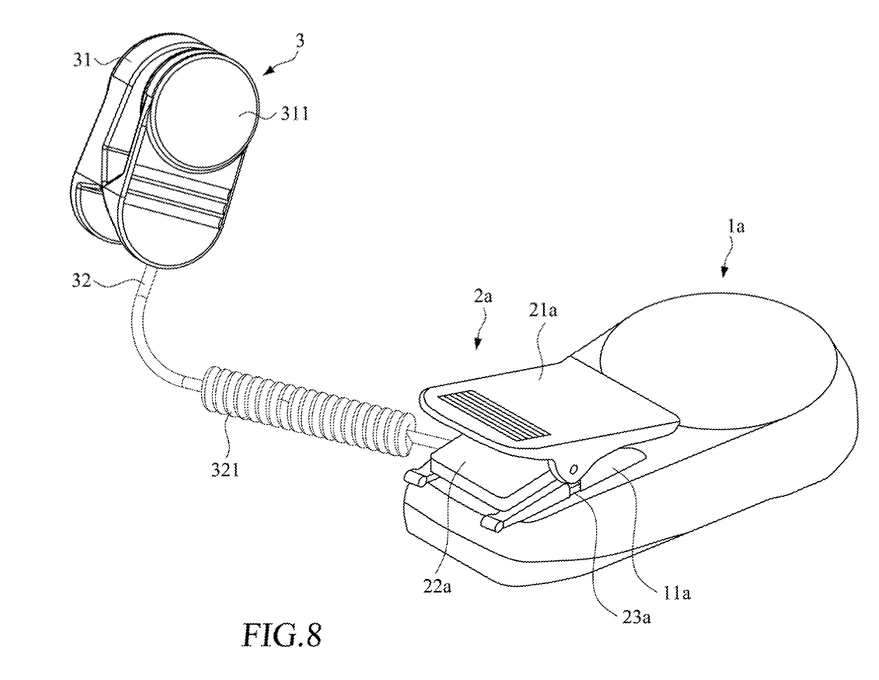

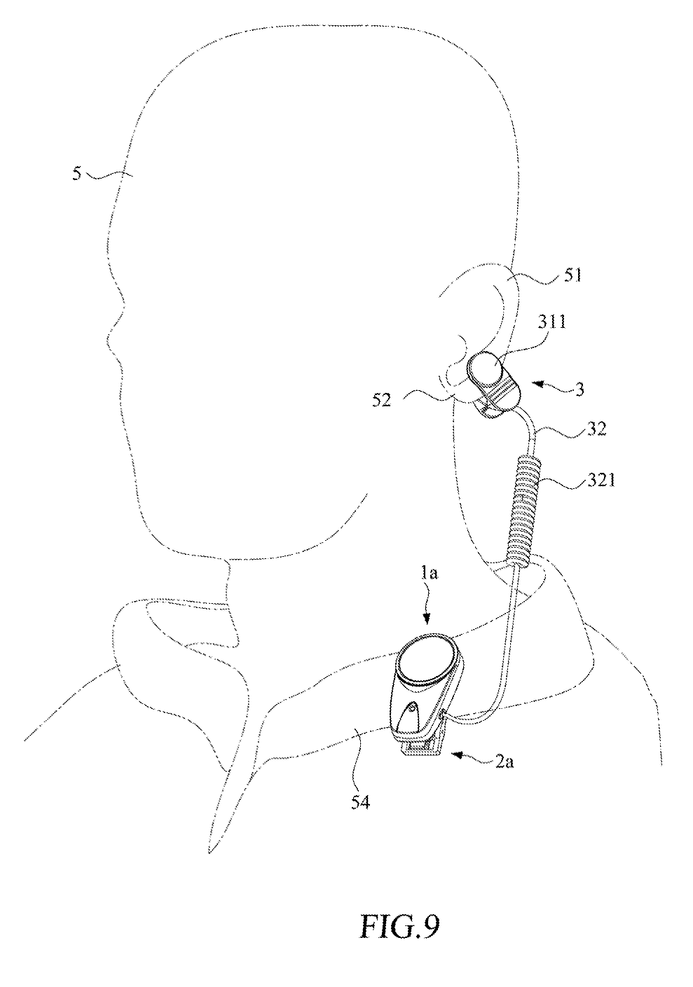

Referring to FIGS. 8-9, FIG. 8 is a perspective view showing a third embodiment according to the present invention and FIG. 9 is a schematic view illustrating an application of the third embodiment of the present invention. An attaching structure 2a comprises an attaching section 21a that is a clip structure. When the attaching structure 2a is coupled to the device body 1a, the coupling component 22a of the attaching structure 2a is coupled to a coupling section 11a of the device body la and a securing element 23a formed at a side of the coupling component 22a engages and secures the coupling section 11a of the device body 1a to have the attaching structure 2a coupled to and fixed to the device body 1a.

A user 5 may clamp the attaching structure 2b to a clothing collar 54 of the user 5 and then clamps the ear clip 3 to the earlobe 52 of the ear 51 of the user 5. Under such an arrangements, the blood flow sensor 311 that is mounted to the ear dip 3 may detect the variation of blood flowrate through a blood capillary of the earlobe 52 of the ear 51 of the user 5 to identify the condition of heart beat of the user 5, which is transmitted through the signal transmission wire 32 to the device body 1a. As such, a user 5 may easily use the present invention.

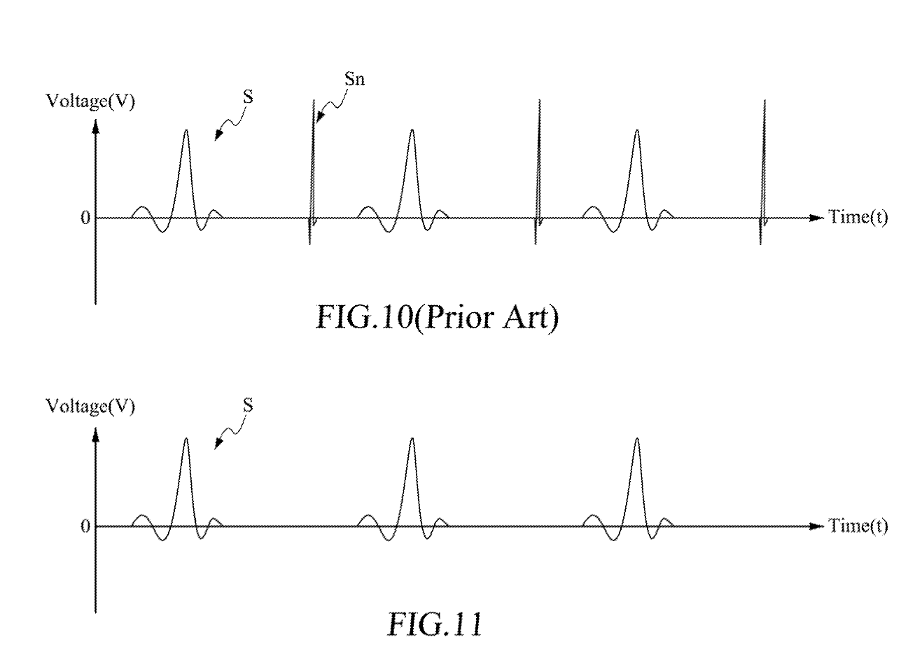

Referring to FIGS. 10 and 11, FIG. 10 is a plot showing shaking signal generated by a conventional ear clip heart beat detection device when the user is taking an exercise. FIG. 11 is a plot showing the shaking signals occurring when a user is taking exercise are eliminated according to the present invention. The conventional ear clip heart beat detection device may easily generate a shaking signal Sn (shown in FIG. 10) when the user is taking an exercise so that measurement of the user's heart beat information S becomes incorrect, making it valueless for reference. The present invention uses an extension assembly to hold the ear clip, so that the ear clip does not easily shake and generate a shaking signal Sn even when the user is taking an exercise to affect the user's heart beat information S. Therefore, correct measurement of the heart beat information S can be obtained for reference by the user.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. An ear-carrying heart beat detection device, comprising:
a device body, which comprise a first coupling section and first pivot joint section;
an attaching structure, which is coupled to the first coupling section of the device body to have the attaching structure coupled to the device body for being worn on a selected position by a user;
an ear clip, which comprises a clamping section, a signal transmission wire, and a second coupling section, the clamping section comprising a blood flowrate sensor mounted thereto and connected with the signal transmission wire for detecting a blood flowrate signal from an earlobe of the user;
an extension assembly, which is connected between the ear clip and the device body so that a relative distance of the ear clip with respect to the device body is adjustable through the extension assembly;
a control circuit, which is arranged in the device body, the control circuit comprising:
a heart beat detection circuit, which is connected by the signal transmission wire to the blood flowrate sensor to receive the blood flowrate signal generated by the blood flowrate sensor and generates in response thereto a heart beat signal, and
a wireless transmission circuit, which is connected to the heart beat detection circuit to receive the heart beat signal and transmit the heart beat signal in a wireless fashion; and
a wrist watch unit, which is worn on a wrist of the user, the wrist watch unit comprising:
a wireless receiver circuit, which receives the heart beat signal transmitted from the wireless transmission circuit,
a processing unit, which is connected to the wireless receiver circuit,
a display unit, which is connected to the processing unit to display the heart beat signal,
a memory unit, which is connected to e processing unit

2. The ear-carrying heart beat detection device as claimed in claim 1, wherein the first pivot joint section of the device body comprises a pivot pin.

3. The ear-carrying heart beat detection device as claimed in claim 1, wherein the extension assembly comprise an extendable bar, the extendable bar comprising a second pivot joint section at one end, the extendable bar being coupled to the second coupling section of the ear clip, the extendable bar being coupled to the first pivot joint section of the device body with the second pivot joint section.

4. The ear-carrying heart beat detection device as claimed in claim 3, wherein the second pivot joint section of the extendable bar comprises a pivot hole that is rotatably coupled to the pivot pin.

5. The ear-carrying heart beat detection device as claimed in claim 3, wherein the extendable bar has an opposite end forming a stop.

6. The ear-carrying heart beat detection device as claimed in claim 1, wherein the second coupling section of the ear clip comprises a clamp.

7. The ear-carrying heart beat detection device as claimed in claim 6, wherein the clamp rotatably clamps on the extendable bar so as to be movable or rotatable with respect to the extendable bar as being subjected to an external force.

8. The ear-carrying heart beat detection device as claimed in claim 1, wherein the first coupling section of the device body and the coupling component of the attaching structure are respectively provided with mateable positioning elements.

9. The ear-carrying heart beat detection device as claimed in claim 8, wherein the positioning elements comprise mateable toothed rings.

10. The ear-carrying heart beat detection device as claimed in claim 1, wherein the attaching structure comprise an attaching section and a coupling component, the coupling component being coupled to the first coupling section of the device body, the attaching section being coupled via the coupling component to the device body, the attaching section being adapted to be worn on an ear of the user.

11. An ear-carrying heart beat detection device, comprising:
a device body, which comprise a first coupling section and a first pivot joint section;
an attaching structure, which is coupled to the first coupling section of the device body to have the attaching structure coupled to the device body for being worn on a selected position by a user;
an ear clip, which comprises a clamping section, a signal transmission wire, and a second coupling section, the clamping section comprising a blood flowrate sensor mounted thereto and connected with the signal transmission wire for detecting a blood flowrate signal from an earlobe of the user, the signal transmission wire comprising at least one extendable section so that a relative distance of the ear clip with respect to the device body is adjustable through the extendable section:
a control circuit, which is arranged in the device body, the control circuit comprising:
a heart beat detection circuit, which is connected by the signal transmission wire to the blood flowrate sensor to receive the blood flowrate signal generated by the blood flowrate sensor and generates in response thereto a heart beat signal, and
a wireless transmission circuit, which is connected to the heart beat detection circuit to receive the heart beat signal and transmit the heart beat signal in a wireless fashion; and
a wrist watch unit, which is worn on a wrist of the user, the wrist watch unit comprising:
a wireless receiver circuit, which receives the heart beat signal transmitted from the wireless transmission circuit,
a processing unit, which is connected to the wireless receiver circuit, a display unit, which is connected to the processing unit to display the heart beat signal, and
a memory unit, which is connected to the processing unit.

12. The ear-carrying heart beat detection device as claimed in claim 11, wherein the first pivot joint section of the device body comprises a pivot pin.

13. The ear-carrying heart beat detection device as claimed in claim 11, wherein the second coupling section of the ear dip comprises a clamp.

14. The ear-carrying heart beat detection device as claimed in claim 11, wherein the attaching structure comprise an attaching section and a coupling component, the coupling component being coupled to the first coupling section of the device body, the attaching section being coupled via the coupling component to the device body, the attaching section being adapted to be worn on an ear of the user.

15. The ear-carrying heart beat detection device as claimed in claim 11, wherein the attaching structure comprises a clip structure that is adapted to clamp on a clothing collar of the user.

16. The ear-carrying heart beat detection device as claimed in claim 11, wherein the first coupling section of the device body and the coupling component of the attaching structure are respectively provided with mateable positioning elements.
